(19) United States
(12) Patent Application Publication
(10) Pub. No.: US 2022/0362371 A1
(43) Pub. Date: Nov. 17, 2022

(54) HUMAN RESPIRATORY SYNCYTIAL VIRUS STRAIN AND ITS USE

(71) Applicant: UNIVERSITEIT ANTWERPEN, Antwerpen (BE)

(72) Inventors: Peter DELPUTTE, Kortrijk (BE); Winke VAN DER GUCHT, Wilrijk (BE)

(21) Appl. No.: 17/761,459

(22) PCT Filed: Sep. 21, 2020
(86) PCT No.: PCT/EP2020/076330

§ 371 Date: Mar. 17, 2022

(30) Foreign Application Priority Data
Sep. 20, 2019 (EP) 19198640.5
Sep. 20, 2019 (EP) 19198804.7

(51) Int. Cl. A61K 39/12; A61P 31/14

(52) U.S. Cl. CPC A61K 39/12; A61P 31/14; C12N 2760/18511; C12N 2760/18534

(57) ABSTRACT

An isolated respiratory syncytial virus (RSV) strain is disclosed. Also an immunogenic composition comprising said novel RSV strain is disclosed. Further, the present application is directed to said RSV strain or the immunogenic composition comprising said strain for use in the generation of an immune response against RSV in a subject. Also their use in the diagnosis of an RSV-associated diseases is disclosed.

### CROSS-REFERENCES TO RELATED APPLICATIONS

This application is a national-stage application under 35 U.S.C. § 371 of International Application No. PCT/EP2020/076330, filed Sep. 21, 2020, which International Application claims benefit of priority to European Patent Application No. 19198640.5, filed Sep. 20, 2019, and European Patent Application No. 1918804.7, filed Sep. 20, 2019.

### FIELD OF THE INVENTION

The present invention is directed to a novel isolated respiratory syncytial virus (RSV) strain. Also an immunogenic composition comprising said novel RSV strain is disclosed. Further, the present application is directed to said RSV strain or the immunogenic composition comprising said strain for use in the generation of an immune response against RSV in a subject. Also their use in the diagnosis of an RSV-associated diseases is disclosed.

### BACKGROUND TO THE INVENTION

Respiratory syncytial virus (RSV) infections are the leading cause of serious lower respiratory tract infections in children below the ager of 5 and the elderly [1,2]. No vaccine or antiviral has been licensed to treat RSV infections except Synagis® (Palivizumab) which is a humanized monoclonal antibody that is used for the passive immunization of infants with high risk of developing serious lower respiratory tract infections. RSV vaccine development is hampered by two main reasons: (I) immunopathology that can be caused by vaccination leading to enhanced disease upon primary natural infection and (II) the elicitation of a short-lived weak immune response. Reinfections with RSV are very common whereas yearly epidemics do not indicate significant antigenic changes. Therefore, induction of strong immune responses via vaccination is difficult as well. This is illustrated by several vaccine strategies against RSV that have failed due to lack of efficacy in clinical endpoints, for example the Novavax F vaccine that failed phase III clinical trials.

Of all possible vaccine strategies, live-attenuated and live-vector vaccines have proven not to prime for vaccine enhanced respiratory disease in naïve individuals [3,4]. Most of the published research on antivirals, vaccine development and virus-host interactions are performed with the RSV A2 strain, that was isolated from the population in 1961. This prototype strain has been extensively cultured in immortal cell lines to increase its fitness for cell culture, leading to the divergence and adaptation of the strain to artificial lab environments. Recent clinical RSV isolates are mainly unavailable, are not stable at 37° C. and 4° C. and are difficult to grow the high cell-free virus titers, whereas most RSV virus remains cell-associated [5]. Cell-associated virus is difficult to release from the cell even by additional freeze-thaw cycles leading to inactivation of the virus and high amounts of cell debris contaminating the virus samples.

For the development of live-attenuated vaccines, a high concentration of purified avirulent virus is necessary that contains enough surface proteins for the elicitation of a potent neutralizing antibody response. RSV contains three surface proteins of which two, the G-protein and the F-protein, elicit the main neutralizing antibody response. As the G-protein is the most variable between different virus strains and as it evolves at the highest rates of all RSV proteins, it has changed dramatically since the isolation of laboratory strains RSV A2 (1961) and RSV Long (1956), which have an unclear passage history as well.

Development of RSV vaccines is thus challenging because of safety risks and the fact that RSV has yet unknown mechanisms that hinder the induction of strong, long-lasting and protective immune responses.

In the present application, we have isolated and characterized virus from hospitalized patients and found one isolated (BE/ANT-A11/17) that has a proved increased infectivity and produced high amounts of cell-free virus in HEp-2 cells compared to prototype strain RSV A2. Additionally, thermal stability of the virus is increased at 4° C. compared to other clinical isolates from the same season.

### SUMMARY OF THE INVENTION

The present invention is thus directed to an isolated human respiratory syncytial virus (RSV) strain deposited on Aug. 23, 2019 under accession number LMBP 11505 at the Belgian Co-ordinated Collection of Micro-Organisms (BCCM). In the present application, said RSV strain is also referred to as BE/ANT-A11/17. In a further embodiment, said isolated human RSV strain is typically characterized in that the majority of the virus particles of said isolated RSV strain have a globular morphology. In another embodiment, said isolated RSV strain is typically characterized in that it has an increased thermal stability as compared to the reference RSV strain A2. In yet another embodiment, said isolated human RSV strain is characterized in that it has an increased infection capacity as compared to the reference RSV strain RSV A2.

The present invention also discloses an immunogenic composition comprising the isolated RSV strain BE/ANT-A11/17 according to all its different embodiments. Said immunogenic composition further comprises a pharmaceutically acceptable carrier or excipient.

In another aspect of the present application, the isolated RSV strain BE/ANT-A11/17 or the immunogenic composition comprising the isolated RSV strain BE/ANT-A11/17 according to all their different embodiments for use as a medicine; in particular for use in the treatment and/or prevention of RSV-associated diseases in a subject.

In still another aspect, the isolated RSV strain BE/ANT-A11/17 or the immunogenic composition comprising the isolated RSV strain BE/ANT-A11/17 according to all their different embodiments are for use in the generation of an immune response against RSV in a subject.

In another aspect, the isolated RSV strain BE/ANT-A11/17 or the immunogenic composition comprising the isolated RSV strain BE/ANT-A11/17 according to all their different embodiments are for use in the diagnosis of an RSV-associated disease. For example, production of the isolated RSV strain BE/ANT-A11/17 can be used in diagnostics, for example for the detection antigens.

In another aspect, the isolated RSV strain BE/ANT-A11/17 of the present invention can also be used in virus-neutralization assays. The present application, thus provides the use of the isolated RSV strain BE/ANT-A11/17 in virus-neutralization assays.

In another aspect, the present application provides the use of the isolated RSV strain BE/ANT-A11/17 or of the immunogenic composition comprising the isolated RSV strain BE/ANT-A11/17 according to all their different embodiments, as an antigen to generate an immune response during vaccination. In said context, the isolated RSV strain BE/ANT-A11/17 or the immunogenic composition comprising the isolated RSV strain BE/ANT-A11/17 according to all their different embodiments can thus be used in human immune challenge studies or controlled human infection trials, said studies or trials involving the intentional exposure of the subject to the isolated RSV strain or the immunogenic composition.

In a further embodiment, a monoclonal antibody against the isolated RSV strain BE/ANT-A11/17 according to all its embodiments is disclosed, said monoclonal antibody being for use in the diagnosis of an RSV-associated disease.

The present invention further provides the use of said monoclonal antibody against the isolated RSV strain BE/ANT-A11/17 as an antigen to generate an immune response during vaccination.

In a further aspect, the present application provides a method for eliciting an immune response against RSV, said method comprising administering to a subject an isolated RSV strain BE/ANT-A11/17 or an immunogenic composition comprising the isolated RSV strain BE/ANT-A11/17 according to any of their embodiments disclosed herein. In a further embodiment, said method is typically characterized in that the immune response comprises a protective response that reduces or prevents infection with RSV and/or reduces or prevents a pathological response following infection with an RSV.

In still a further aspect of the present application, an isolated RSV strain or an immunogenic composition comprising one or more isolated RSV strains are provided for use as a medicine, wherein the majority of the virus particles of said one or more isolated RSV strains have a globular morphology. In a further embodiment, an isolated RSV strain or an immunogenic composition comprising one or more isolated RSV strains are provided for use in the treatment and/or prevention of RSV-associated diseases in a subject. In still a further embodiment, the isolated RSV strain BE/ANT-A11/17 is one of said one or more isolated RSV strains. In still another embodiment, said isolated human RSV strain or the immunogenic composition comprising one or more isolated RSV strains for use as a medicine or for use in the treatment and/or prevention of RSV-associated diseases in a subject is characterized in that said one or more isolated RSV strains have an increased thermal stability and/or an increased infection capacity as compared to the reference strain RSV A2.

In another aspect, an isolated RSV strain or an immunogenic composition comprising one or more isolated RSV strains are provided for use in the generation of an immune response against RSV in a subject wherein the majority of the virus particles of said one or more isolated RSV strains have a globular morphology. In still a further embodiment, the isolated RSV strain BE/ANT-A11/17 is one of said one or more isolated RSV strains. In still another embodiment, said isolated human RSV strain or the immunogenic composition comprising one or more isolated RSV strains for use in the generation of an immune response against RSV in a subject is characterized in that said one or more isolated RSV strains have an increased thermal stability and/or an increased infection capacity as compared to the reference strain RSV A2. In all its aspects, an immune response is further defined as to comprise a protective response that reduces or prevents infection with RSV and/or reduces or prevents pathological response following infection with an RSV. The present application further discloses a method for eliciting an immune response against RSV, said method comprising administering to a subject an isolated RSV strain or an immunogenic composition wherein the majority of the virus particles of said one or more isolated RSV strains have a globular morphology. In a further embodiment, said immune response comprises a protective response that reduces or prevents infection with RSV and/or reduces a pathological response following infection with an RSV.

In a specific aspect, the isolated RSV strain or the immunogenic composition according to their different embodiments is for use in human challenge studies or controlled human infection trials, said studies or trials involving the intentional exposure of the subject to the isolated RSV strain or the immunogenic composition.

In a further aspect, the subject as disclosed in all different embodiments of the invention is a mammal; preferably a human. In an even more preferred embodiment, this subject is a child or an infant. In another embodiment, the subject is an adult. In still a further embodiment, the subject is an older adult.

In another aspect, the subject of the present invention can already be diagnosed with an RSV infection. In another aspect, the subject of the present invention is not yet diagnosed with an RSV infection. In still another aspect, the subject of the present invention is not yet diagnosed with an RSV infection but shows the clinical symptoms of an RSV infection.

The RSV infection can be an acute RSV infection. In another embodiment, the RSV infection is a chronic RSV infection.

In another aspect, the RSV infection is selected from pharyngitis, croup, bronchiolitis, pneumonia, or a combination thereof. In still another aspect, the RSV infection is selected from acute pharyngitis, acute croup, acute bronchiolitis, acute pneumonia, or a combination thereof. In still another embodiment, the RSV infection is selected from chronic pharyngitis, chronic croup, chronic bronchiolitis, chronic pneumonia, or a combination thereof.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIGS. 1A-1B: Phylogenetic trees for RSV-A and RSV-B clinical isolates. The phylogenetic trees were constructed with maximum-likelihood with 1000 bootstrap replicates using MEGA X software. The trees are based on a 342nt and 330nt fragment of the G protein of RSV-A (FIG. 1A) and RSV-B (FIG. 1B) strains respectively, consisting of the second hypervariable region. Nucleotide sequences of the clinical isolates (indicated with ⋅) were compared to reference strains found on GenBank (indicated with genotype and accession number). The outgroups are represented by prototype strains M11486 for RSV-A and M17213 for RSV-B. Bootstrap values greater than 70% are indicated at the branch nodes and the scale bare represents the number of substitutions per site.

FIGS. 2A-2D: Growth kinetics and infectious virus production in HEp-2 cells. (FIG. 2A-2B) HEp-2 cells were infected with clinical isolates and RSV reference strains A2 and B1. Cultures were fixed after 24 h, 48 h and 72 h, permeabilized and stained with polyclonal antibody (pAb) goat-anti-RSV antibody and AF488 donkey-anti-goat (IgG). Nuclei were visualized with DAPI and cultures were analyzed with fluorescence microscopy. RSV positive cells were counted and calculated to the total number of nuclei to reach a percentage of RSV infected cells. (FIG. 2A) Growth kinetics of RSV-A clinical isolates and (FIG. 2B) Growth kinetics of RSV-B clinical isolates. (FIGS. 2C-2D) HEp-2 cells were infected with clinical isolates and RSV reference strains A2 and B1. After 24 h, 48 h and 72 h, supernatants were collected and used for quantification by conventional plaque assay. Data represents mean values ±SEM (N=3), significant differences compared to the reference strains are indicated by *p<0.05; ***p<0.001 (two-way ANOVA).

FIGS. 3A-3D: Growth kinetics and infectious virus production in A549 cells. (FIGS. 3A-3B) A549 cells were infected with clinical isolates and RSV reference strains A2 and B1. Cultures were fixed after 24 h, 48 h and 72 h, permeabilized and stained with pAb goat-anti-RSV antibody and AF488 donkey-anti-goat (IgG). Nuclei were visualized with DAPI and cultures were analyzed with fluorescence microscopy. RSV positive cells were counted and calculated to the total number of nuclei to reach a percentage of RSV infected cells. (FIG. 3A) Growth kinetics of RSV-A clinical isolates and (FIG. 3B) Growth kinetics of RSV-B clinical isolates. (FIGS. 3C-3D) A549 cells were infected with clinical isolates and RSV reference strains A2 and B1. After 24 h, 48 h and 72 h, supernatants were collected and used for quantification by conventional plaque assay. Data represents mean values ±SEM (N=3), significant differences compared to the reference strains are indicated by *p<0.05; ***p<0.001 (two-way ANOVA).

FIGS. 4A-4D: Growth kinetics and infectious virus production in BEAS-2B cells. (FIGS. 4A-4B) BEAS-2B cells were infected with clinical isolates and RSV reference strains A2 and B1. Cultures were fixed after 24 h, 48 h and 72 h, permeabilized and stained with pAb goat-anti-RSV antibody and AF488 donkey-anti-goat (IgG). Nuclei were visualized with DAPI and cultures were analyzed with fluorescence microscopy. RSV positive cells were counted and calculated to the total number of nuclei to reach a percentage of RSV infected cells. (FIG. 4A) Growth kinetics of RSV-A clinical isolates and (FIG. 4B) Growth kinetics of RSV-B clinical isolates. (FIGS. 4C-4D) BEAS-2B cells were infected with clinical isolates and RSV reference strains A2 and B1. After 24 h, 48 h and 72 h, supernatants were collected and used for quantification by conventional plaque assay. Data represents mean values ±SEM (N=3), significant differences compared to the reference strains are indicated by *p<0.05;**p<0.01 ***;p<0.001 (two-way ANOVA).

FIGS. 5A-5F: Thermal stability profiles at 37° C., 32° C. and 4° C. Clinical isolates, RSV A2 and RSV B1 were aliquoted and exposed to 37° C. (FIGS. 5A-5B), 32° C. (FIGS. 5C-5D) or 4° C. (FIGS. 5E-5F). One aliquot of each was snap frozen at 0 h, 24 h, 48 h and 72 h. Aliquots were used for quantification by conventional plaque assay and calculated to the amount at 0 h. Data represents mean values ±SEM (N=3), significant differences compared to the reference strains are indicated by *p<0.05;**p<0.01; ***p<0.001 (two-way ANOVA).

FIGS. 6A-6D: The capacity for syncytia formation of clinical isolates. HEp-2 cells were infected with clinical isolates and RSV reference strains A2 and B1 for 2 h, inoculum was replaced by DMEM-10 containing 0.6% Avicel® and incubated for 48 h at 37° C. Afterwards, cells were fixed, permeabilized and stained with pAb goat-anti-RSV and AF488 donkey-anti-goat. Nuclei were visualized with DAPI and cultures were analyzed with fluorescence microscopy. (FIGS. 6A-6B) Mean syncytium size was calculated by counting the number of nuclei in syncytia in three pictures taken at 10× magnification. (FIGS. 6C-6D) Mean syncytium frequency was calculated by dividing the number of syncytial cells by the total number of infected cells. Data represents mean values ±SEM (N=3), significant differences compared to the reference strains are indicated by *p<0.05; ***p<0.001 (one-way ANOVA).

FIG. 7: Plaque reduction of the clinical isolates with palivizumab. HEp-2 cells were infected for 2 h with clinical isolates and reference strains that were pre-incubated for 1 h with a palivizumab dilution series. Inoculum was replaced with DMEM-10 containing 0.6% Avicel® and incubated for three days at 37° C. Afterwards, the cells were fixed, stained with palivizumab as primary antibody and goat-anti-human conjugated with HRP, plaques were visualized with chloronapthol. Individual values are plotted as 2log EC50, data represents mean values ±SD (N=3).

FIGS. 8A-8G: mRNA levels of mucins 1, 4, 5AC and 5B in infected 549 cells. A549 cells were infected with an MOI of 0.1 of clinical isolates and reference strains for 2 h at 37° C. Inoculum was replaced with DMEM-10 and cells were incubated for 48 h at 37° C. Afterwards, cells were lysed, total RNA was extracted and the expression of MUC1 (FIG. 8A), MUC4 (FIG. 8B), MUC5AC (FIG. 8C), MUC5B (FIG. 8D), MUC2 (FIG. 8E), MUC6 (FIG. 8F) and MUC13 (FIG. 8G) was determined by qRT-PCR. Data represents mean values ±SEM (N=3), statistically significant differences compared to the reference strains are indicated with ***p<0.001 (one-way ANOVA).

FIG. 9: Correlation between MUC13 mRNA expression and “Resvinet” score. Pearson's correlation was used to determine the relationship between the variables ‘relative MUC13 mRNA expression’ and ‘Resvinet socre’.

### DETAILED DESCRIPTION OF THE INVENTION

### Definitions

In the context of the present application, “diagnosis” and “diagnosing” generally includes a determination of a subject's susceptibility to a disease or disorder, a determination as to whether a subject is presently affected by a disease or disorder.

The terms “prognosis” and “prognose” refer to the act or art of foretelling the course of a disease. Additionally, the terms refer to the prospect of survival and recovery from a disease as anticipated from the usual course of that disease or indicated by special features of the individual case.

The term “severity” of a disease refers to the extent of an organ system derangement or physiologic decompensation for a patient. It gives a medical classification such as minor, moderate, major and extreme. The severity of a disease is used to provide a basis for evaluating hospital resource use and to establish patient care guidelines.

The terms “treatment”, “treating”, “treat” and the like refer to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” covers any treatment of a disease in a mammal, in particular a human, and includes: (a) preventing the disease or symptom from occurring in a subject which may be predisposed to the disease or symptom but has not yet been diagnosed as having it; (b) inhibiting the disease symptoms, i.e. arresting its development; or (c) relieving the disease symptom, i.e. causing regression of the disease or symptom.

The term “biological sample” encompasses a variety of fluid samples, including blood and other liquid samples of biological origin, or tissue samples, or mixed fluid-cell or mixed fluid-tissue samples, obtained from an organism that may be used in a diagnostic or monitoring assay. The term specifically encompasses a clinical fluid or tissue sample, and further includes cell supernatants, cell lysates, serum, plasma, urine, amniotic fluid, biological fluids, tissue biopsies, lavages, aspirates, sputum or mucus. The term also encompasses samples that have been manipulated in any way after procurement, such as treatment with reagents, solubilization, or enrichment for certain components.

In the present application, the inventors identified a novel RSV strain, further referred to as BE/ANT-A11/17. Said strain was deposited on Aug. 23, 2019, at the Belgian Co-ordinated Collection of Micro-Organisms (BCCM) with accession number LMBP 11505.

Strain BE/ANT-A11/17 has been isolated from the population in 2017 and has a clear increased stability at 4° C. and 37° C. compared to other clinical RSV isolates. The strain easily infects immortal cell lines to high virus titers and has been observed producing high titers of cell-free virus. The production of high amounts of cell-free virus combined with increased stability at 37° C. and 4° C. has the potential to ease the production of virus for vaccine production and/or for diagnostics development.

The virus strain RSV A2 is currently primarily used for RSV releted research. The virus strain is isolated in 1961 and has adapted to immortal cell culture through extensive passaging since its isolation. The virus produces high amounts of cell-free but also cell-associated virus, resulting in contamination of cell debris after purification. This virus is homogenous in form and size [6], increasing the difficulty to purify enough virus for experiments and vaccine development. Additionally, RSV is very thermolabile and is deactivated very quickly at higher temperatures. The F protein has been implicated in thermal stability [4] and specific mutations have been identified increasing thermal stability [7]. Virus strain BE/ANT-A11/17 has been isolated recently in 2017 and has been shown to produce high amounts of cell-free virus. Additionally, the virus remains more stable than other clinical isolates, and does not contain any of the mutations increasing thermal stability as described earlier [7]. Virus produced from BE/ANT-A11/17 infected cells also has a more uniform phenotype and is fully released from the infected cell (not cell-associated), thus making it possible to collect virus with less cell debris after purification. The inventors also found that the majority of the virus particles of the BE/ANT-A11/17 strain have a globular morphology, which might contribute to the increased thermal stability and increased infection capacity of the BE/ANT-A11/17 strain.

The invention is now further described using the following examples.

### EXAMPLES

### A. Identification and Characterization of RSV Strains, Including BE-ANT-A11/17

### Materials and Methods

### Cells and Viruses

The HEp-2, A549 and Vero cell lines were obtained from and cultured to the instructions of ATCC. BEAS-2B cell line was a generous gift from dr. Ultan F. Power (Queens University Belfast, Ireland). All cells were cultured in Dulbecco's modified Eagle medium containing 10% inactivated fetal bovine serum (DMEM¹⁰) (Thermo Fisher Scientific). RSV reference strains A2 and B1 were obtained from BEI resources, RSV A2 was cultivated in HEp-2 cells as described by Van der Gucht W. et al [8] and RSV B1 was cultivated on Vero cells in medium containing 2% inactivated fetal bovine serum (iFBS) until cytopathic effect (CPE) was visible throughout the flask. Virus was collected as described for A2 and quantified in a conventional plaque assay on HEp-2 as described by Schepens B. et al [9]. Briefly, HEp-2 cells were seeded at a concentration of 175 000 cells/ml in clear 96 well plates (Falcon) 1 day prior to infection. Cells were washed with DMEM without iFBS (DMEM⁰) and infected with 50 μl of a 1/10 dilution series made in DMEM⁰. Cells were incubated for 2 h at 37° C. after which the inoculum was replaced by DMEM¹⁰ containing 0.6% avicel (FMC biopolymer) and incubated for 3 additional days at 37° C., 5% CO₂. Afterwards, cells were washed with PBS, fixed with 4% paraformaldehyde solution and stained with palivizumab (leftovers provided by the department of Pediatrics, Antwerp University Hospital) and goat-anti human secondary IgG conjugated with horseradish peroxidase (HRP) (Thermo Fisher Scientific) and visualized using chloronaphtol solution (Thermo Fisher Scientific).

### Virus Isolation from Clinical Samples

This study was approved by the ethical committee of the Antwerp University Hospital and the University of Antwerp (16/46/491). Mucus was collected from children showing symptoms of an RSV-related bronchiolitis during the winter seasons of 2016-2017 and 2017-2018 after parental consent was given. The mucus was extracted by a nasal swab and/or a nasopharyngeal aspirate, which were stored at 4° C. for less than 10 h. One day prior to mucosal extraction, HEp-2 cells were seeded at a concentration of 175 000 cells/ml in a clear 96 well plate (Falcon). Samples were vortexed for 1 minute with glass beads (Sigma-Aldrich) before inoculating HEp-2 cells with 50 μl of a 1:4 dilution series of the sample, made in DMEM without iFBS (DMEM⁰). After 2 h of incubation with the inoculum, 50 μl of DMEM containing iFBS, antibiotics (penicillin/streptomycin (life technologies), moxifloxacin (Sigma-Aldrich)) and anti-fungals (Fungizone)(Sigma-aldrich) was added to obtain a final concentration of DMEM with 2% FBS. Plates were incubated for 7 days at 37° C. and 5% CO₂. After 7 days, the plates were checked for syncytia formation and 50 μl of the well with the lowest concentration of original sample but still presenting CPE, was transferred to a newly seeded plate, following the same protocol. After another 7 days, the wells were rechecked for syncytia formation. A total of 250 μl from wells with syncytia was transferred to a freshly seeded T25, which was left until cytopathic effects were visible throughout the flask. Supernatant was collected, centrifuged for 10 min at 1000×g, aliquoted, snap frozen in liquid nitrogen and labelled passage 0. Virus obtained from these clinical samples was propagated until passage 3 on HEp-2 cells to obtain a plaque forming unit (PFU) high enough to perform the following experiments. One sample did not propagate efficiently on HEp-2 cells and was propagated for 3 passages on Vero cells until a high enough PFU was reached.

### RSV-A and RSV-B Subtyping

RNA for subtyping was extracted from passage 0 virus using the QIamp viral RNA extraction mini kit (QIAgen) following the manufacturer's instructions. A multiplex reaction mix was made with superscript III platinum one-step quantitative kit (Thermo Fisher Scientific) in a final volume of 25 μl containing 5 μl RNA, 12,5 μl PCR master mix, 1 μl superscript RT/Platinum Taq polymerase and 2.5 μl of a pre-mixed primer/probe solution. This solution contains a final concentration of 5 μM of each primer and 1 μM of each probe. The primers for RSV-A are located in the L gene (RSVQA1: 5′-GCT CTT AGC AAA GTC AAG TTG AAT GA-3′ (SEQ ID No: 1) and RSVQA2: 5′-TGC TCC GTT GGA TGG TGT AAT-3′ (SEQ ID No: 2), RSVQA probe: 5′-HEX/ACA CTC AAC AAA GAT CAA CTT CTG TCA TCC AGC-′3- IABkFQ (SEQ ID No: 3) wherein ZEN is inserted after ACA CTC AAC in the probe) and the primers for RSV-B are located in the N gene (RSVQB1: 5′-GAT GGC TCT TAG CAA AGT CAA GTT AA-3′ (SEQ ID No: 4) and RSVQB2: 5′-TGT CAA TAT TAT CTC CTG TAC TAC GTT GAA-3′ (SEQ ID No: 5), RSVQB probe: 5′-RTEX615/TGA TAC ATT AAA TAA GGA TCA GCT GCT GTC ATC CA-′3-BHQ_2 (SEQ ID No: 6)). Reaction was run on a Real-time PCR machine (Stratagene, Mx3000P, Thermo Fisher Scientific) with the following program: 50° C. for 30 min, 94° C. for 5 min followed by 45 cycles of 15 s at 94° C. and 1 min at 55° C. Ct values below 40 were counted as positive.

### Nucleotide Sequencing and Phylogenetic Analysis

Viral RNA was extracted using the QIAmp viral RNA mini kit (Qiagen) according to the instructions provided by the manufacturer. Viral RNA of the G-gene was transcribed to cDNA and amplified using the One-step RT-PCR kit (Qiagen) and the following primers as described by L. Houspie et al. [10]. For RSV-A, the forward primer G267FW (5′ ATG CAA CAA GCC AGA TCA AG 3′ (SEQ ID No: 7) and reverse primer F164RV (5′ GTT ATC ACA CTG GTA TAC CAA CC 3′ (SEQ ID No: 8)) were used, for RSV-B, the forward primer BGF (5′ GCA GCC ATA ATA TTC ATC ATC TCT 3′ (SEQ ID No: 9)) and reverse primer BGR (5′ TGC CCC AGR TTT AAT TTC GTT C 3′ (SEQ ID No: 10)) were used. Primers were added to the reaction mix consisting of 10 μl 5× RT-PCR buffer, 2 μl dNTP, 2 ml enzyme, 20 μl H₂O to a final concentration of 30 pmol. 10 μl RNA extract was added to the reaction mix. The PCR was performed in a thermocycler (Unocycler, VWR) following the given program: 30 min at 50° C. for the Reverse Transcription step, 15 min at 95° C. for PCR activation, 40 amplification cycles consisting of 30 s at 95° C., 1 min at 55° C. and 1 min at 72° C. followed by a final extension step for 10 min at 72° C. The amplified cDNA was subjected to a 1% agarose gel electrophoresis, visualized with Gelgreen™ (VWR) to determine the length. Amplified cDNA was delivered to the VIB Genetic service facility (University of Antwerp) for PCR cleanup and DNA sequencing with the following primers as described by L. Houspie et al [10]: in addition to the PCR amplification primers, for RSV-A: G516R (5′ GCT GCA GGG TAC AAA GTT GAA C 3′ (SEQ ID No: 11)) and G284F (5′ ACC TGA CCC AGA ATC CCC AG 3′ (SEQ ID No: 12)) and for RSV-B: BGF3 (5′ AGA GAC CCA AAA ACA CYA GCC AA 3′ (SEQ ID No: 13)) and BGR3 (5′ ACA GGG AAC GAA GTT GAA CAC TTC A 3′ (SEQ ID No: 14)) were provided for sequencing. Sequences were annotated in Snapgene and contigs were built in Bioedit with the CAP3 application. Multiple sequence alignments from reference strains and contigs and phylogenetic trees were constructed in MEGA X using the maximum likelihood method.

### Viral Replication Kinetics

HEp-2, A549 and BEAS-2B cells were seeded at a concentration of 175 000 cells/ml in black CELLSTAR® 96 well plates with a μclear® flat bottom suitable for fluorescence microscopy (Greiner-bio one) 1 day prior to inoculation. Briefly before inoculation, the cells were washed with DMEM⁰, followed by inoculation. Clinical RSV and RSV-A2 were diluted to infect the cells at a multiplicity of infection (MOI) of 0.01. Virus was left to adhere for 2 h at 37° C., 5% CO₂ and replaced with DMEM¹⁰. Cells were fixed with 4% paraformaldehyde after 24 h, 48 h and 72 h, permeabilized and stained with palivizumab followed by goat anti-human secondary antibody conjugated with Alexa Fluor 488 (AF488) (Thermo Fisher Scientific) and additional DAPI nucleus staining (Sigma-Aldrich).

### Infectious Virus Production

HEp-2, A549 and BEAS-2B cells were seeded at a concentration of 200 000 cells/ml in 24 well plates 24 h prior to infection. Briefly, before infection, cells were washed with DMEM⁰ and afterwards infected with clinical isolates and RSV A2 and RSV B1 at an MOI of 0.01. Supernatant was collected after 24 h, 48 h and 72 h, aliquoted, snap frozen and stored at −80° C. Supernatant was quantified using a conventional plaque assay on HEp-2 cells as described above.

### Thermal Stability Assay

Aliquots of clinical isolates and RSV A2 and RSV B1 were thawed and diluted in DMEM° to obtain a starting concentration of 1×10⁵ PFU/ml and re-aliquoted. Immediately after aliquotation, one aliquot of each sample was snap frozen in liquid nitrogen as T0. The other aliquots were stored at 4° C., at 32° C. or at 37° C. for 24 h, 48 h and 72 h, snap frozen in liquid nitrogen and stored at −80° C. until quantification was performed. A conventional plaque assay on HEp-2 cells as described earlier was used to quantify the remaining PFU in each aliquot.

### Cell-to-Cell Fusion Assay

24 h prior to inoculation, HEp-2 cells were seeded at a concentration of 175 000 cells/ml in black CELLSTAR® 96 well plates with a μclear® flat bottom suitable for fluorescence microscopy (Greiner bio-one). Cells were inoculated with clinical RSV and RSV-A2 at a MOI of 0.05 for 2 h at 37° C., 5% CO₂. After 2 hours, the inoculum was removed and replaced by DMEM¹⁰ containing 0.6% Avicel (FMC biopolymer). After 48 h cells were washed with PBS, fixed with 4% paraformaldehyde solution, permeabilized and stained with palivizumab followed by goat anti-human secondary antibody conjugated with AF488 (Thermo Fisher Scientific). DAPI staining was performed to stain the nuclei (Sigma-Aldrich).

### Plaque Reduction Assay

The plaque reduction assay was performed as described by Leemans A. et al [11]. Briefly, HEp-2 cells were seeded at a concentration of 175 000 cells/ml in a clear 96 well plate (Falcon) 24 h prior to inoculation. Palivizumab was diluted 1:40 and further in a 1:2 dilution series, which was incubated with diluted virus for 1 h at 37° C., 5% CO₂. Afterwards, the cells were washed briefly with DMEM⁰, and inoculated with 50 μl of the virus-antibody solution for 2 h at 37° C., 5% CO₂. Then, the inoculum was replaced with DMEM¹⁰ containing 0,6% avicel (FMC biopolymer). The plates were incubated for 3 days at 37° C., 5% CO₂, washed with PBS and fixed with 4% paraformaldehyde solution. The cells were permeabilized, stained with palivizumab antibody followed by goat anti-human IgG conjugated with horseradish peroxidase (HRP) and colored using chloronaphtol solution (Thermo Fisher Scientific).

### Mucin mRNA Expression Assay

A549 cells were seeded at a concentration of 200 000 cells/ml in 24 well plates 24 h prior to inoculation (Greiner bio-one). Cells were infected with a MOI of 0.1 for 2 h at 37° C., 5% CO₂. After 2 h, inoculum was replaced by DMEM¹⁰ and was incubated for an additional 48 h. After 48 h, cell supernatant was collected, spun down at 1000×g for 15 minutes and only the pellet was kept. The still adherent cells were lysed with lysis buffer from the nucleospin kit (MN) and added to the pellet. The solution was pipettet up and down several times and frozen at −80° C. until extraction was performed. RNA isolation was done following manufacturer's instructions of the nucleospin RNA kit (MN). Concentrations were evaluated using the Nanodrop® (Thermo Fisher Scientific) and 1 μg of RNA was used to convert to cDNA using the SensiFast™ cDNA synthesis kit (Bioline). Relative gene expression was determined with the GoTaq qPCR master mix (Promega) with SYBR Green Fluorescence detection on a QuantStudie 3 Real-time PCR instrument (Thermo Fisher Scientific). Standard QuantiTect primers available from Qiagen were used for GAPDH (QT00079247), β-actin (QT00095431), MUC1 (QT00015379), MUC4 (QT00045479), MUC5AC (QT00088991) and MUC5B (QT01322818). Analysis and quality control were performed using qbase+ software (Biogazelle), relative expression of the target genes was normalized to the expression of the housekeeping genes GAPDH and β-actin.

### F-Gene Nucleotide Sequencing

Viral RNA of the F-gene was transcribed to cDNA and amplified using the One-step RT-PCR kit (Qiagen) and the following primers as described by L. Tapia et al. [12]. For the F-gene, four primers were used in pairs to transcribe and amplify the F-gene in two segments, F1 and F2. For F1, the forward primer RSVAB_F1FW (5′ GGC AAA TAA CAA TGG AGT TG 3′ (SEQ ID No: 15)) and reverse primer RSVAB_F1RV (5′ AAG AAA GAT ACT GAT CCT G 3′ (SEQ ID No: 16)) were used. For F2 the forward primer RSVAB_F2FW (5′ TCA ATG ATA TGC CTA TAA CA 3′ (SEQ ID No: 17)) and RSVAB_F2RV (5′ GGA CAT TAC AAA TAA TTA TGA C 3′ (SEQ ID No: 18)) were used. Both primer sets are the same for RSV-A and RSV-B strains. Primers were added to the reaction mix consisting of 10 μl 5× RT-PCR buffer, 2 μl dNTP, 2 ml enzyme, 20 μl H₂O to a final concentration of 30 pmol. 10 μl RNA extract was added to the reaction mix. The PCR was performed in a thermocycler (Unocycler, VWR) with the program: 30 min at 50° C. for the RT step, 15 min at 95° C. for PCR activation, five amplification cycles consisting of 30 s at 95° C., 30 s at 48° C. and 1 min at 72° C. followed by 35 amplification cycles consisting of 30 s at 95° C., 30 s at 55° C. and 1 min at 72° C., and a final extension step for 10 min at 72° C. The length of the amplified cDNA was verified with 1% agarose gel electrophoresis and visualized with Gelgreen™ (VWR). Amplified cDNA was delivered to the VIB Neuromics support facility (University of Antwerp) for PCR cleanup and DNA sequencing with the same primers. Sequences were annotated in SnapGene and contigs were built in BioEdit with the CAP3 application. Multiple sequence alignments from contigs were constructed in MEGA X using Muscle.

### Fluorescence Microscopy and Image Analysis

Fluorescence photographs were acquired using an Axio Observer inverted microscope and a Compact Light source HXP 120C with filter set 49, 10 and 20 for blue, green and red fluorophores respectively (Zeiss). Image analysis was done using Zeiss ZEN 2.3 blue edition imaging software and ImageJ version 2.0.0-rc-43/1.50e. Calculations were made in Excel for Mac and Graphpad Prism 6.

### Statistical Analysis

Data for viral growth kinetics, infectious virus production and thermal stability are presented as means (±SEM) of the indicated independent repeats. To determine the significance between the clinical isolates and the reference (A2 or B1), data was analyzed with a two-way ANOVA. Fusion data and MUC expression represents means (±SEM), significance was calculated between the clinical isolates and their references with a one-way ANOVA. Data for plaque reduction represents means (±SD), significance was calculated between clinical isolates and references with a one-way ANOVA. Calculations were done using Graphpad Prism 6.

### Results

### Clinical Samples and Detection of RSV

Nasal swabs and nasopharyngeal aspirates were obtained from one patient in December of 2016 and from 24 patients between October and January 2017-2018. RSV-A was detected in one sample of 2016-2017 and in 11 samples of 2017-2018. RSV-B was also detected in 11 samples of the 2017-2018 season. Of the remaining two RSV-negative samples, one tested positive for human metapneumovirus (hMPV), one remained negative for RSV, hMPV and Rhinovirus 1. HEp-2 cells were infected with the samples on the day of the aspiration of secretions or the day afterwards, without freezing the samples. After two weeks of incubation, 11 samples did not result in syncytia formation or positive fluorescent staining in either the nasal swab culture or the aspirate culture and were therefore not used in any of the following assays. Cultures that showed syncytia formation were used to grow the virus on HEp-2 cells. One sample was further grown on Vero cells since no significant titers could be reached growing the virus on HEp-2 cells. Said strain was deposited on Aug. 23, 2019, at the Belgian Co-ordinated Collection of Micro-Organisms (BCCM) with accession number LMBP 11505.

TABLE 1
Overview of clinical isolates and viruses used in experiments,
with subtyping results and cell type used for propagation

| NAME: | SUBTYPE: | GROWN ON: |
|---|---|---|
| BE/ANT-A1/16 | RSV-A | HEp-2 |
| BE/ANT-B2/17 | RSV-B | HEp-2 |
| BE/ANT-A7/17 | RSV-A | HEp-2 |
| BE/ANT-A8/17 | RSV-A | HEp-2 |
| BE/ANT-A10/17 | RSV-A | HEp-2 |
| BE/ANT-A11/17 | RSV-A | HEp-2 |
| BE/ANT-A12/17 | RSV-A | HEp-2 |
| BE/ANT-B13/17 | RSV-B | HEp-2 |
| BE/ANT-B15/17 | RSV-B | HEp-2 |
| BE/ANT-A18/17 | RSV-A | HEp-2 |
| BE/ANT-B20/17 | RSV-B | Vero |
| BE/ANT-A21/17 | RSV-A | HEp-2 |
| RSV A2 | RSV-A | HEp-2 |
| RSV B1 | RSV-B | Vero |

### Phylogenetic Analysis

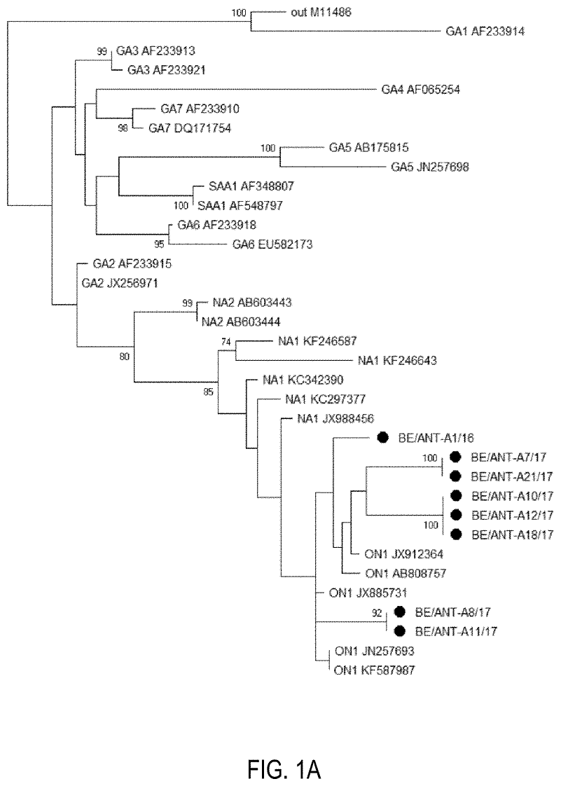

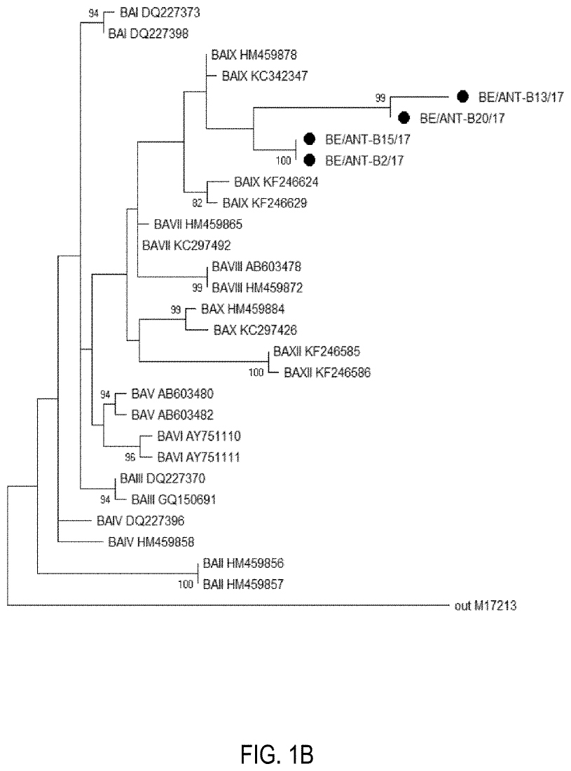

Sequences of the G-gene of all samples were obtained and aligned with previously reported representative sequences from GenBank. The phylogenetic trees of RSV-A and RSV-B sequences were setup (FIG. 1).

All RSV-A sequences cluster within the ON1 genotype that contains a 72 nt duplication and all RSV-B sequences contain a 60 nt duplication in the G-gene, assigning them to the BA genotype, further differentiated into the BAIX genotype.

### G Protein Sequence Analysis

The nucleotide sequence of the G-gene of each clinical isolate was determined and translated to their corresponding in-frame protein sequences by aligning them to the RSV A2 protein sequence in GenBank. Sequences were annotated to the corresponding domains of the G protein sequence: the N-terminal domain (NT), the transmembrane domain (TM), both mucus-like regions (MLR), the central conserved domain (CCD) and the heparin binding domain (HBD). All sequences of recent RSV-A clinical isolates differ from the RSV A2 sequence in 32 amino acids, all spread throughout both MLRs, confirming the use of these regions in phylogeny studies (data not shown). Clinical isolates differ from each other as well in 19 amino acid residues. BE/ANT-A1/16 contains three unique amino acids that are not found in the other clinical isolates, whereas mutations in the clinical isolates obtained in the winter of 2017 are also observed in other clinical isolates. Analysis indicated that sequences of BE/ANT-A7/17 and BE/ANT-A21/17 are very much alike, as well as the G protein sequences of BE/ANT-A10/17, BE/ANT-A12/17 and BE/ANT-A18/17. Sequences BE/ANT-A8/17 and BE/ANT-A11/17 are also very similar, which is indicated by the phylogenetic analysis. The 72 nt duplication in the MLR-II is present in all clinical isolates starting from amino acid residue 204 to residue 207. Sequences of RSV-B isolates are aligned to the sequence of RSV B1 and all clinical isolates differ from RSV B1 in 21 residues spread out through the MRLs (data not shown). Ten residues are different between the clinical isolates themselves, mainly in the MLRs but also in the HBD. All isolates contain the 60 nt duplication in the MLR-II and a sequence deletion of three residues at the end of MLR-I. Sequences of BE/ANT-B2/17 and BE/ANT-B15/17 are mainly similar, as are BE/ANT-B13/17 and BE/ANT-B20/17.

### Viral Replication Kinetics

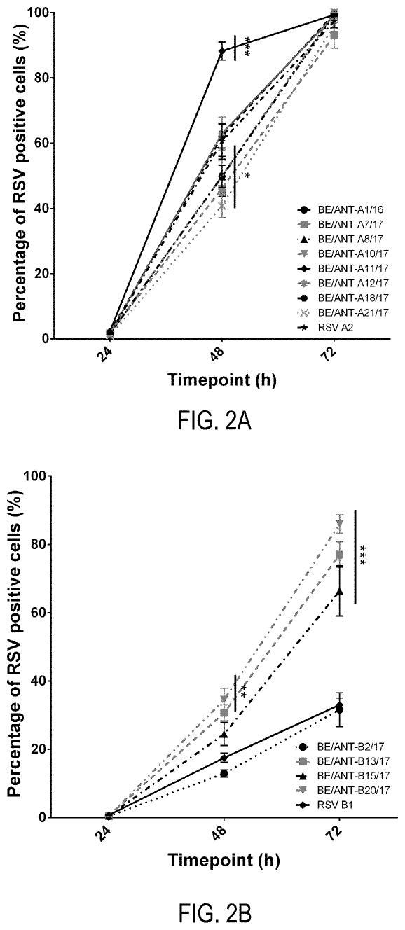

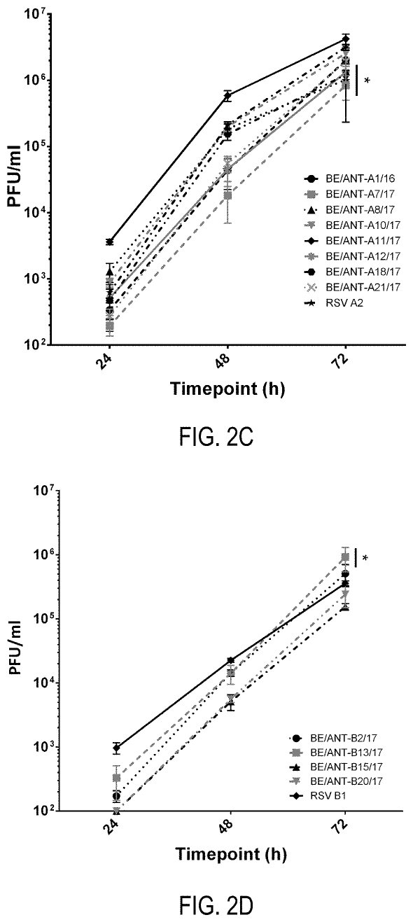

To study the dynamics of viral infection, viral replication kinetics and infectious virus production were assessed in HEp-2, A549 and BEAS-2B cells. Cells were infected for 24 h, 48 h and 72 h with a MOI of 0.01, fixed, fluorescently stained and analyzed with fluorescence microscopy to evaluate viral replication kinetics. Infectious virus production was evaluated through the collection of supernatants after 24 h, 48 h and 72 h post-infection with an MOI of 0.01. Supernatant was snap frozen and used for quantification through plaque assay. Viral replication kinetics in HEp-2 cells for RSV-A (FIG. 2A) strains yielded one strain (BE/ANT-A11/17) that resulted in significantly higher percentages of RSV-infected cells after 48 h compared to RSV A2. The BE/ANT-A11/17 also produced more infectious virus particles after 24 h post inoculation (p.i.) compared to all other strains (FIG. 2C). Three strains (BE/ANT-A21/17, BE/ANT-A7/17, BE/ANT-A8/17) replicated more slowly than the RSV A2 at 48 h but a fully infected culture was observed after 72 h of infection. The RSV-B strains (FIG. 2B and D) showed two strains grown on HEp-2 cells (BE/ANT-B13/17, BE/ANT-B15/17) and one strain grown on Vero cells (BE/ANT-B20/17) that resulted in significantly more infected cells at 72 h than the reference B1, whereas just one strain (BE/ANT-B2/17) seemed to result in comparable infection as the B1. Infectious virus production of RSV-B shows that even though the BE/ANT-B20/17 and BE/ANT-B15/17 reach a very high percentage of infected cells, significantly less infectious particles are produced compared to the other strains, suggesting that the particles may not be efficiently released in the supernatant and remain more cell-associated.

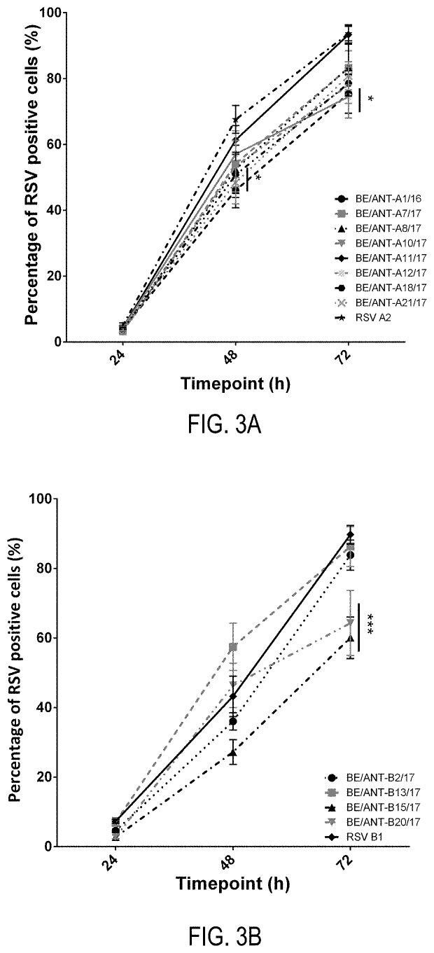

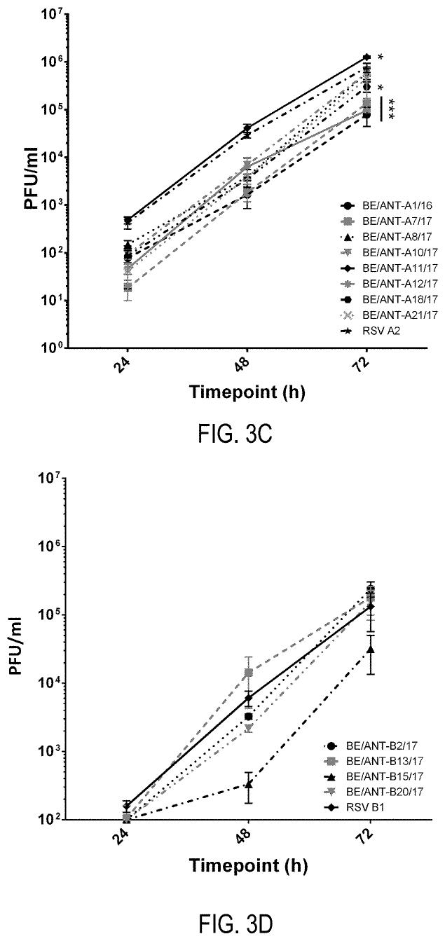

The same experiment was repeated in the A549 (FIG. 3) cell line in which for the RSV-A isolates (FIG. 3A), the RSV A2 shows the highest percentage of infected cells, followed closely by the BE/ANT-A11/17, performing only slightly less than in the HEp-2 cells. The aforementioned strains also were the ones that produced the highest amounts of infectious virus in A549 cells (FIG. 3C). Whereas in HEp-2 cells both the BE/ANT-B13/17 and BE/ANT-B20/17 isolates perform better than the RSV B1, results of A549 replication kinetics suggest that the BE/ANT-B13/17 and BE/ANT-B2/17 strains reach similar infection rates (FIG. 3B). The BE/ANT-B20/17 reached about 50% infection after 48 h but the infection then seemed to flatten out towards 72 h, resulting in a significant difference with infection rates of the RSV B1. Interestingly, the isolate BE/ANT-B2/17, which did not efficiently infected HEp-2 cells now reached a near 100% infection in 72 h. Unsurprisingly, the BE/ANT-B15/17 achieved again the lowest number of infectied cells and levels of virus production in A549 cells (FIG. 3D).

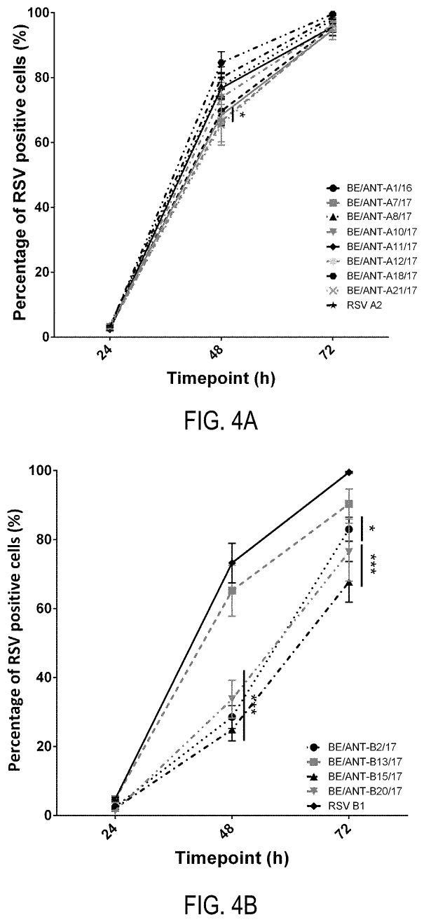

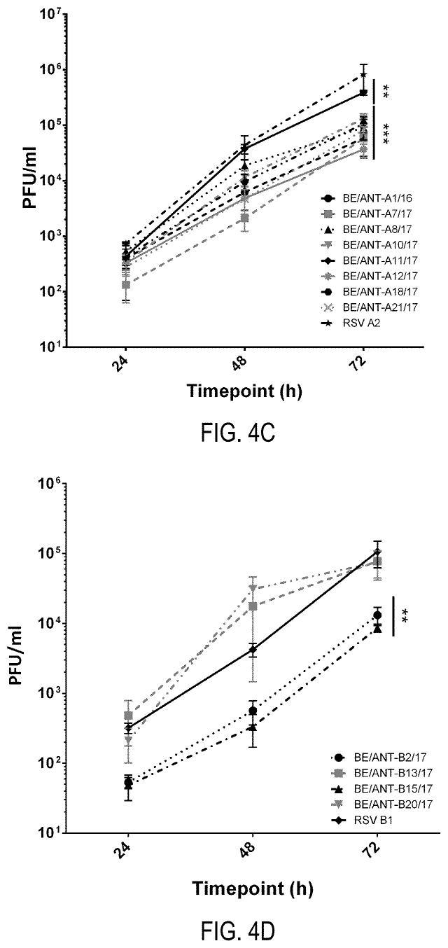

As the BEAS-2B cell line is also a highly permissive cell line for RSV infection and widely used, we also assessed viral growth and production kinetics in this cell line (FIG. 4). For all RSV-A clinical isolates, no major differences were observed after 48 h and 72 h of infection in percentage of infected cells (FIG. 4A). After 72 h of infection, the amount of viable particles released by the cells was the highest for RSV A2 and clinical isolate BE/ANT-A11/17. Larger differences were observed between the clinical isolates of the RSV-B subtype (FIG. 4B). BE/ANT-B13/17 reached percentages and viable particle production that were comparable to RSV-B1 (FIGS. 4B and 4D). Isolates BE/ANT-B2/17 and BE/ANT-B15/17 had the lowest infection rates and infectious virus production in both this cell line as well as in the HEp-2 cells (FIGS. 4B and 4D).

Overall, clinical isolate BE/ANT-A11/17 replicated very efficiently in all cell lines, and remarkably, achieving even higher infection rates in the HEp-2 cell line than the RSV A2. Also, two clinical isolates of the RSV-B (BE/ANT-B20/17 and BE/ANT-B13/17) replicated very well in HEp-2 and A549 cell lines and quite well in BEAS-2B. Overall, differences in infection kinetics were observed within the different clinical isolates.

### Thermal Stability

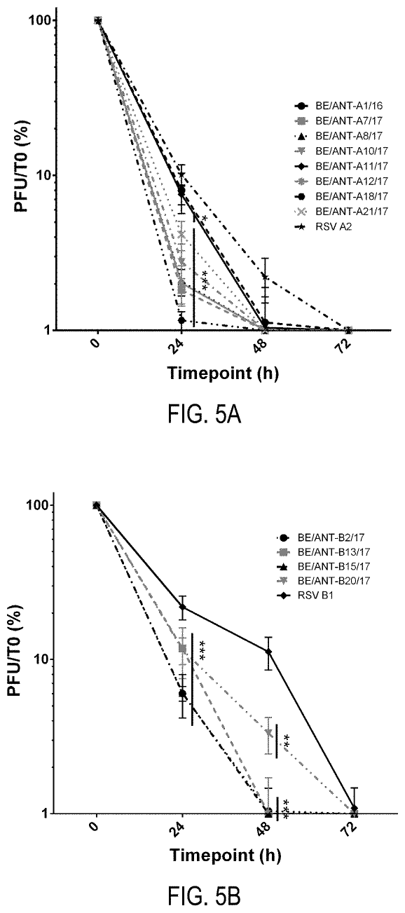

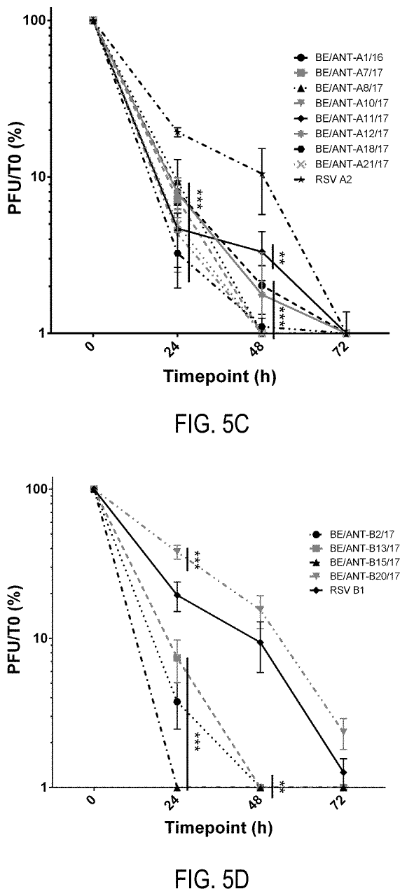

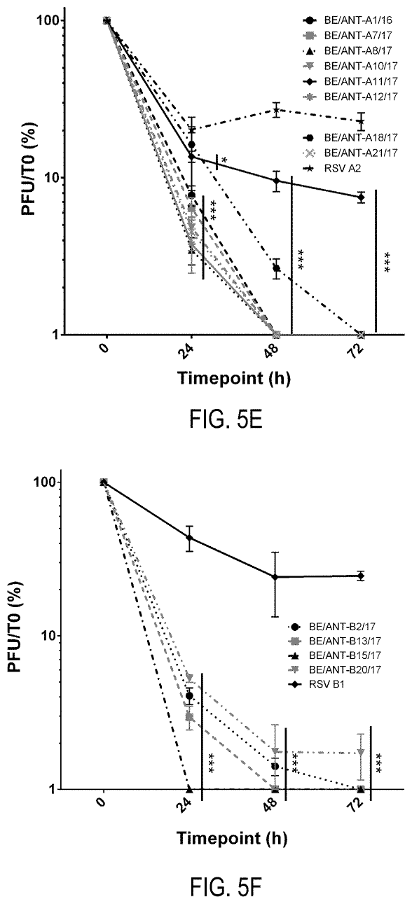

Differences in the F protein are shown to be involved in thermal stability of viral particles [13]. Aliquots of each virus containing 1×10⁵ PFU/ml were incubated at three different temperatures: 37° C. (in vitro incubator temperature and core body temperature) (FIGS. 5A and B), 32° C. (upper airway temperature) (FIGS. 5C and D) and 4° C. (storage temperature) (FIGS. 5E and F) for 24 h, 48 h and 72 h. Aliquots were snap frozen in liquid nitrogen and used for conventional plaque assay to quantify infectious virus. For all RSV-A isolates and RSV A2, higher temperatures were associated with a faster decay of infectious virus. Curiously, BE/ANT-A11/17 conserved higher PFU at 4° C. than other RSV-A isolates although at the other temperatures there was no difference. Also BE/ANT-A18/17 was preserved slightly better at 4° C., however at 72 h no viable virus was detected. RSV-B isolate BE/ANT-B20/17 retained higher titers for the duration of the experiment compared to other RSV-B isolates but its overall stability was less than the reference RSV B1. The only exception is at 32° C., where its viral titers remained higher than RSV B1. Isolate BE/ANT-B15/17 seems to decay especially fast at any other temperature than 37° C.

### Cell to Cell Fusion

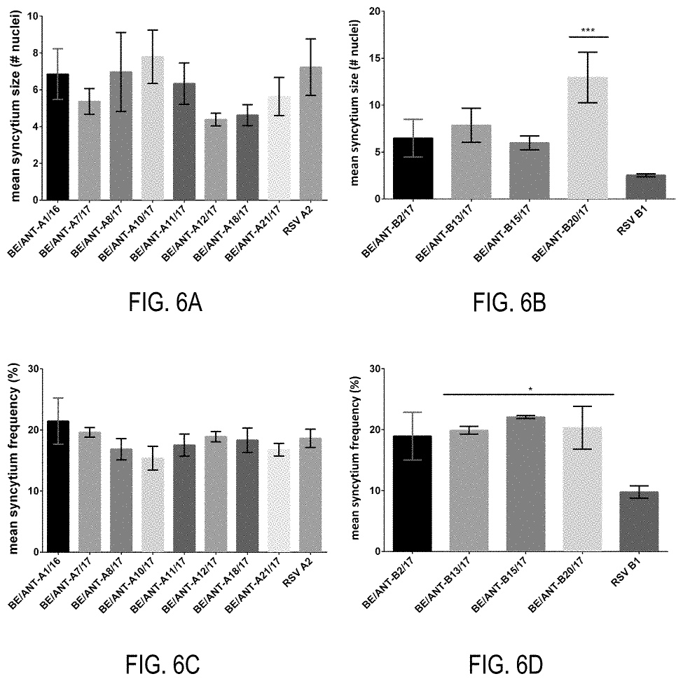

Syncytia formation has long been considered a typical characteristic of RSV infection in immortal cell lines, and it has been used as a measure of activity of the fusion protein [14]. HEp-2 cells were infected with an MOI of 0.05 and incubated for 48 h with an overlay of DMEM¹⁰ containing 0.6% avicel to allow spreading of the infection to neighboring cells only. Afterwards, cells were fixed, fluorescently stained and analyzed with fluorescence microscopy. Mean syncytium size was determined, (FIGS. 6A and 6B) as well as mean syncytium frequency (FIGS. 6C and 6D) by counting the number of nuclei belonging to syncytia relative to the total number of nuclei of infected cells. Mean syncytium size of all RSV-A clinical isolates (FIG. 6A) lies between four and seven nuclei per cell, with BE/ANT-A1/16, BE/ANT-A8/17 and BE/ANT-A10/17 having the largest syncytia. The smallest syncytia were produced by BE/ANT-A12/17. Mean syncytium frequencies lie between 16% and 21%, with the lowest frequency found for BE/ANT-A10/17, which suggested that it promotes the formation of larger syncytia rather than many small syncytia (FIG. 6C). Clinical isolate BE/ANT-B20/17 formed significantly larger syncytia with a mean size of 13 compared to all clinical isolates (FIG. 6B). Reference strain RSV B1 formed almost no syncytia, with the smallest size and lowest frequency of all viruses tested.

### Plaque Reduction by Palivizumab

Viral neutralization by palivizumab was assessed with a conventional plaque reduction assay. Virus was incubated with a two-fold dilution series of palivizumab for 1 h at 37° C. and then transferred to HEp-2 cells for 2 h at 37° C. to allow infection by non-neutralized virus. Afterwards, the supernatant was replaced by DMEM¹⁰ containing 0.6% avicel and incubated for three days until plaques were visible to the naked eye. Plaques were counted to determine the concentration of palivizumab in which 50% of the virus was neutralized.

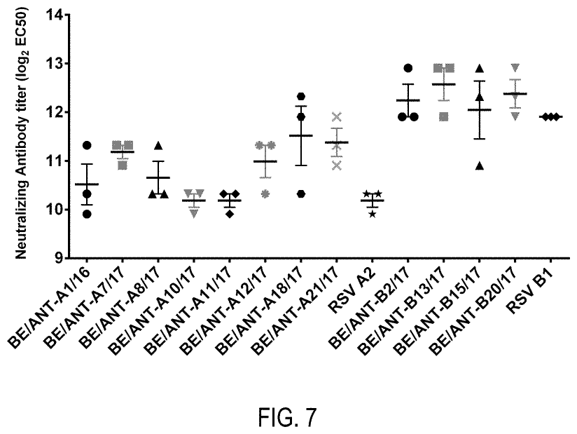

FIG. 7 shows that RSV-A clinical isolates BE/ANT-A7/17 and BE/ANT-A21/17 are 50% neutralized at lower palivizumab concentrations than most of the other clinical isolates and RSV A2, resulting in better neutralization than the other isolates. Remarkably, RSV A2 and RSV B1 neutralization was significantly different, with palivizumab neutralizing the RSV-B strains much better than the RSV-A strains. Overall no significant differences were observed between RSV-B clinical isolates and the reference RSV-Bl for palivizumab neutralization.

### Mucin Expression

RSV infection is hallmarked by an increase of mucus production and impaired mucociliary clearance. As MUCSAC and MUCSB are important players in the secreted airway mucins and MUC1 and MUC4 in the cell-tethered mucins [15], their mRNA expression levels upon RSV infection of A549 cells was tested. mRNA expression levels of the mucins were assessed by infecting A549 cells for 48 h with an MOI of 0.1, followed by qRT-PCR with primers for the different mucin encoding genes. A549 cells were incubated with virus of each isolate for 2 h, after which the inoculum was removed and replaced with DMEM¹⁰. Cells were incubated for 48 h, collected for lysis followed by an RNA extraction and qRT-PCR.

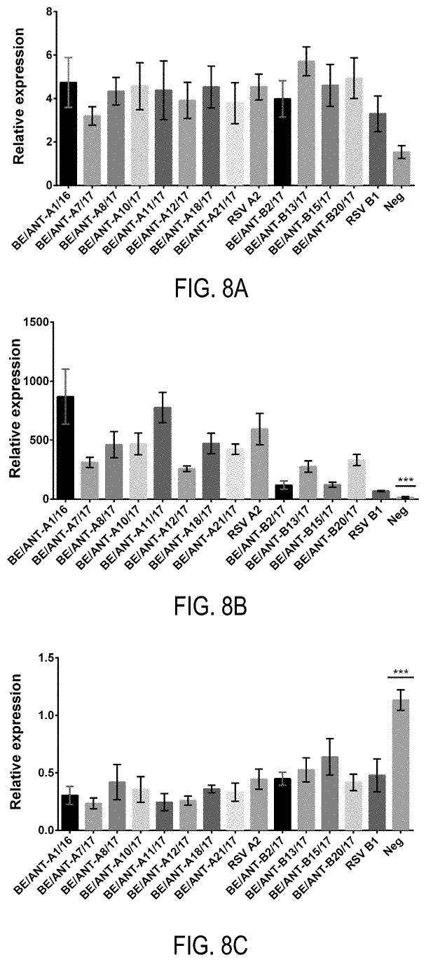

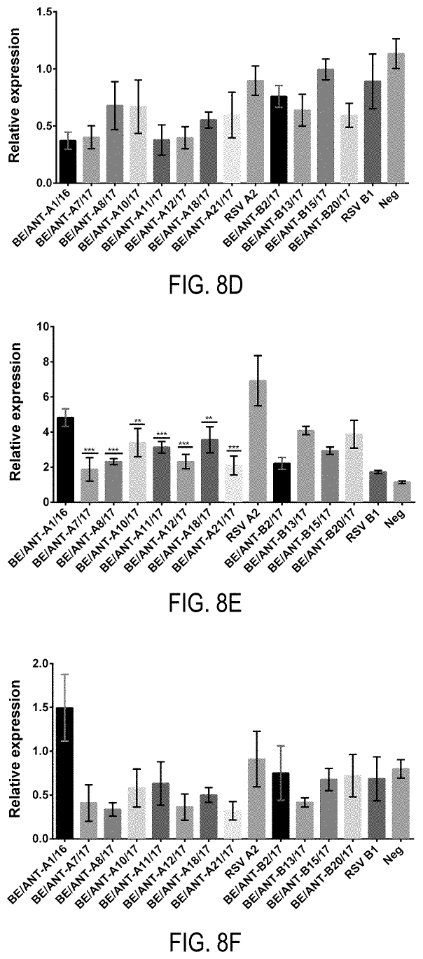

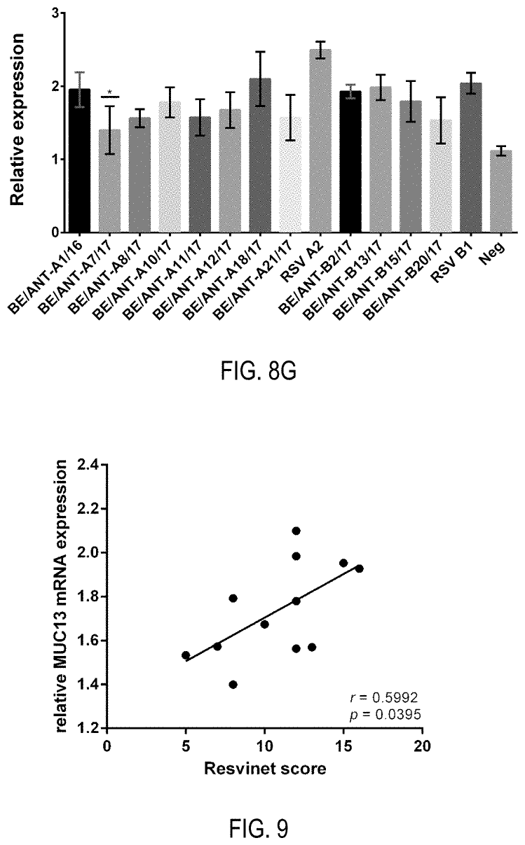

For all clinical isolates and controls, the relative expression of cell-tethered MUC1 (FIG. 8A) is increased compared to the non-infected control. No significant differences can however be observed between RSV isolates and controls.

Expression profiles of the cell-tethered MUC4 show a considerable relative increase compared to the negative control (FIG. 8B). Infection of BE/ANT-A1/16 and BE/ANT-A11/17 resulted in the highest relative increases of MUC4 mRNA among all the RSV-A clinical isolates, whereas BE/ANT-A7/17 and BE/ANT-A12/17 resulted in the lowest increase. For the RSV-B clinical isolates, significantly lower increases are observed when compared to the RSV-A clinical isolates, but an increase is still observed. Infection of isolates BE/ANT-B13/17 and BE/ANT-B20/17 resulted in the highest increase of MUC4 mRNA expression among the RSV-B isolates.

MUC5AC is mainly produced in the epithelial goblet cells, and was previously reported to slightly decrease in A549 cells under the influence of an RSV-infection after 48 h [16]. Here, expression of MUC5AC is significantly reduced upon infection with all clinical isolate infections and reference strains, however no significant differences can be observed between the clinical isolates (FIG. 8C).

MUC5B is produced by surface secretory cells throughout the airways and submucosal glands. Our results show that MUC5B expression is downregulated as a result of RSV infection, with strongest downregulation of RSV-A clinical isolates BE/ANT-A1/16, BE/ANT-A7/17, BE/ANT-A11/17 and BE/ANT-A12/17. Overall downregulation of MUC5B by the RSV-B clinical isolates is limited, with almost none in infections with BE/ANT-B15/17 (FIG. 8D).

MUC2 expression in A549 cells is overall increased for all RSV infections in comparison to the negative control (FIG. 8E). The expression in RSV-A clinical isolates is significantly different from the RSV A2 prototype strains in BE/ANT-A7/17, BE/ANT-A8/17, BE/ANT-A10/17, BE/ANT-A11/17, BE/ANT-A12/17, BE/ANT-A18/17 and BE/ANT-A21/17. No significant differences can be observed between the RSV-B clinical isolates and the RSV B1 prototype strain.

No significant differences in relative expression of MUC6 can be observed between the clinical isolates and their corresponding prototype strains (FIG. 8F). Expression MUC6 as a result of RSV-A clinical isolates results in a relative decrease compared to the prototype strain RSV A2 and the negative control, except for BE/ANT-A1/16.

Relative expression of MUC13 is generally increased for all clinical isolates and prototype strains compared to the negative control (FIG. 8G). Clinical isolates BE/ANT-A7/17 is significantly decreased compared to prototype strain RSV A2, whereas for all other clinical isolates, no significant differences can be observed compared to the corresponding prototype strains.

### Correlation of Mucin Expression with Clinical Symptoms

mRNA expression levels of the mucins were assessed by infecting A549 cells for 48 h with an MOI of 0.1, followed by qRT-PCR with primers for the different mucin encoding genes. A549 cells were incubated with virus of each isolate for 2 h, after which the inoculum was removed and replaced with DMEM¹⁰. Cells were incubated for 48 h, collected for lysis followed by an RNA extraction and qRT-PCR.

Pearson's correlation was used to determine the relationship between MUC13 mRNA expression and the “Resvinet score”. Said Resvinet score [17] is a clinical scale based on seven parameters (feeding intolerance, medical intervention, respiratory difficulty, respiratory frequency, apnoea, general condition, fever) that were assigned different values (from 0 to 3) for a total of 20 points. The correlation coefficient r was 0.5992 with a p value of 0.0395 (FIG. 9), indicating a positive and linear correlation between the two variables. This analysis thus indicates that the relative mRNA expression of MUC13 in respiratory epithelial cells is positively correlated with RSV disease severity, represented by the Resvinet score.

### F Protein Sequence Analysis

As the F protein regulates the most important function of viral entry, the fusion event, differences in its protein sequence are important to map as well. We sequenced the F-gene of each clinical isolate and translated the coding sequences to their corresponding in-frame protein sequence by aligning them to the corresponding RSV A2 and RSV B1 reference strain. All F proteins of the RSV-A clinical isolates differ from the RSV A2 strains in 12 amino acids, three in the signal peptide, three in the F2 subunit, one residue in the fusion peptide, three in the F1 subunit and one in the HRB and transmembrane domain respectively (data not shown). Between the clinical isolates, several differences can be observed, resulting in all unique F protein sequences.

Compared to the RSV A2 sequences, there are two additional potential N-glycosylation consensus sites present in certain RSV-A clinical isolates compared to RSV A2. In the p27 at residue 122, the substitution of A to T in clinical isolates BE/ANT-A1/17, BE/ANT-A8/17, BE/ANT-A10/17, BE/ANT-A11/17, BE/ANT-A12/17 and BE/ANT-A18/17 results in the consensus sequence N-X-T/S indicating a potential N-glycosylation site, which has been previously seen in other clinical isolates and the RSV Long strain. The remaining two clinical isolates BE/ANT-A7/17 and BE/ANT-A21/17 contain a mutation at residue 120 from an N to a S, effectively removing one N-glycosylation site. Two clinical isolates, BE/ANT-A10/17 and BE/ANT-A12/17 have an additional substitution of an I residue to an N at residue 195, forming a new N-glycosylation consensus sequence, which has never been described before.

### B. Identification of Genomic Mutations Leading to the Enhanced Phenotype through Whole Genome Sequencing

Whole genome sequencing will be used to provide an overview of all mutations in the genome of BE/ANT-A11/17 and other strains that may have an effect on the specified phenotype of increased growth, thermal stability and the production of more virus particles. We will especially look to mutations in the M gene, the M2.1 gene and the P gene as these are key players in the assembly and budding process, however, all mutations will be taken into account. To rule out mutations that are part of the evolution of RSV, mutations in the genome of BE/ANT-A11/17 that reoccur in other recent strains that do no exhibit this phenotype, will be ruled out.

### Whole Genome Sequencing

RNA will be extracted from the recent clinical isolates using the QIAmp viral RNA mini kit (Qiagen) following manufacturer's instructions except for the addition of carrier RNA. Instead of 5.6 μl of carrier RNA, only 2.8 μl will be added. Host cell DNA will be removed from the samples with DNase I (Thermo Fisher Scientific) during an incubation step of 15 min at room temperature. The reaction will be stopped with EDTA combined with an incubation step at 65° C. for 10 min. Afterwards, a retrotranscription step with Superscript III reverse transcriptase (Thermo Fisher scientific) will result in random cDNA strands by using SISPA-A primers. Second strand synthesis will be performed with DNA polymerase I (NEB) and the resulting dsDNA will be amplified using SISPA-B primers with a GoTaq polymerase (Promega). The resulting amplified dsDNA will be purified with the DNA clean&concentrator kit (Zymo Research) following the instructions of the manufacturer. Ds cDNA will be eluted with 12 μl of Tris-HCl with a pH of 7.5-8.5 to maximize the performance of the elution. The DNA will be used for a library preparation using the Illumina Nextera flex kit (Illumina) following the instructions of the manufacturer, followed by a sample cleanup with 40 μl Agencourt AMPure XP beads (Beckman-coulter) to recover fragments of 300 bp. The libraries will be quantified with the NEBNext Library Quant kit (Illumina). NGS sequencing will be performed with a 300c MiSeq reagent kit v2 for pair-end sequencing (Illumina) on a Illumina MiSeq Next generation sequencer (Illumina).

### Data Analysis and Mutation Selection

Quality of the fastq files will be evaluated by the FastQC tool. The PrinSeq tool will be used to remove poor quality base calls by trimming 15 nt in both the 5′ and 3′ end. The resulting reads will be filtered with a minimum quality value of 30. Genome mapping will be performed using BWA software in two steps. First, the most appropriate reference sequence will be selected from previously published sequencing in GenBank (NCBI). Secondly, the sequence with the highest genome coverage will be selected as the optimal reference for final mapping. Duplicated reads will be deleted and consensus sequence will be obtained from the BAM file by using markdup module from SAMtools. Host rRNA contamination will be determined by mapping the filtered reads to human cytoplasmic and mitochondrial rRNA databases in GenBank (NCBI). Host DNA contamination will be evaluated with Kraken software.

### C. Site Directed Mutagenesis, Cloning and Recovery of Mutants

### Development and Recovery of Mutants

Selected mutations will be added to standardized RSV gene sequences and will be synthesized by Genscript and delivered in pUC57 simple. The sequences will be subcloned into the vector pSynkRSV-Line19 with appropriate restriction enzymes and T4 DNA ligase (New England Biolabs). Ligation products will be transformed into electrocompetent E. coli cells and plasmid DNA will be recovered using PureLink® HiPure Plasmid Midiprep Kit according to the manufacturer's instructions (Thermo Fisher Scientific). The sequences of the recombinant vectors will be confirmed by DNA sequencing (VIB Neuromics Service Facility, University of Antwerp). Recombinant virus will be recovered as described previously [18]. Briefly, BSR T7/5 cells will be passaged with 1 mg/ml geneticin (Thermo Fisher Scientific) and will be seeded in 6 well plates to be confluent at the time of transfection. BAC constructs and helperplasmids pcDNA 3.1 containing all RSV proteins and Lipofectamine 2000 (Thermo Fisher Scientific) will be diluted in Opti-MEM (Thermo Fisher Scientific) and mixed. After 20 min. incubation, transfection complexes of 600 μl will be added to the cells, incubated for 2 h at room temperature on a shaking plate and further incubated with 600 μl GMEM supplemented with 3% iFBS overnight. Then, transfection complexes will be replaced by medium and sub-passed in T25 flasks two days post-transfection. Every 2 or 3 days, the cells will be subcultured until cytopathic effects were evident throughout the flask. Cells will be scraped and the supernatant will be cleared by centrifugation and snap frozen. Subconfluent HEp-2 cultures will be used to propagate recovered virus for three passages to minimize adaptations to the HEp-2 cells. Virus stocks will be titrated by conventional plaque assay in HEp-2 cells. The presence of the mutations in the viruses will be confirmed by RNA extraction using the QIAmp viral RNA mini kit (Qiagen), followed by a reverse-transcriptase PCR (Agilent Technologies) which was further analyzed by sequencing (VIB Neuromics Service Facility, University of Antwerp).

### D. Evaluation of Mutants in Comparison to Original

### Characterization of Recovered Mutants

The recovered mutants will be evaluated for their infectivity, production of cell-free virus and thermal stability in comparison to the original BE/ANT-A11/17 virus and prototype strains using the following assays:

Growth kinetics in HEp-2 cells: This will allow the evaluation of viral infection, and the comparison of growth and infection speeds of the different mutants. HEp-2 cells will be infected with an MOI of 0.01 and incubated for 24 h, 48 h and 72 h. Afterwards, cells will be fixed with 4% paraformaldehyde, stained with primary polyclonal goat-anti-RSV antibody (Virostat) and secondary Donkey-anti-goat conjugated with Alexa fluor 488 (Thermo fisher scientific) and DAPI. Cultures will be analyzed using the Axio observer fluorescence microscope (Zeiss) with HXP 120C compact light source (Zeiss) and filter sets 49, 10 and 20.

Infectious virus production in HEp-2 cells: This will allow the quantification of cell-free virus during a productive infection. HEp-2 cells will be infected with an MOI of 0.01 and incubated for 24 h, 48 h and 72 h. Cell supernatant will be collected at each time point and quantified using a conventional plaque assay.

Thermal stability of mutants: This technique allows the evaluation of the stability of the viruses at different temperatures. Temperatures of 37° C. (body temperature), 32° C. (estimated nasal temperature) room temperature and 4° C. (vaccine storage temperature) will be tested. Virus will be aliquoted at a quantity of 1*10⁵ PFU/ml and stored for 12 h, 24 h, 48 h, 72 h and 96 h at the given temperatures. Afterwards, aliquots will be snap frozen and quantified using a conventional plaque assay

Transmission electron microscopy of fixed virus and infected cultures: This will allow for the evaluation of the shape and size of cell-free virus particles produced by infected cells when using fixed virus, whereas the evaluation of infected cultures will indicate the amount of cell-associated virus and its size and shape. Virus will be dried to the surface of Permanox slides (Nunc, Lab-tek) overnight and HEp-2 cells will be infected with an MOI of 1 and incubated for 48 h to allow a productive infection. This is followed by a fixation in 0.1M sodium cacodylate-buffered, pH 7.4, 2.5% glutaraldehyde solution for 4 h at 4° C. The slides are rinsed 3 times in 0.1M sodium cacodylate, pH 7.4 (Sigma Aldrich) containing 7.5% saccharose (Sigma-Aldrich) before post-fixation in 1% OSO4 solution (Sigma Aldrich) for 2 h. Dehydration is performed in an ethanol gradient (50%, 70%, 90%, 96%, 2×100%) before embedding the slides in EM-bed812 (Electron microscopy sciences). Ultrathin sections were stained with lead citrate and examined in a Tecnai G2 spirit Bio twin Microscope (FEI) at 120 kV.

Purification and density determination of virus particles produced: This will allow for the evaluation of changes in shape, size and weight of the majority of cell-free virus particles. HEp-2 cells will be infected with an MOI of 0.5 and incubated for 48 h. Supernatant will be collected and cleared of cell debris by centrifugation (10 min 1000×g). Supernatant will be transferred to ultracentrifugation grade tubes (Beckman-coulter) and centrifuged for 90 min at 20 000 rpm in a SW32 rotor (Beckman-Coulter) with an Optima XPN-100 ultracentrifuge (Beckman-coulter). Supernatant will be discarded after this centrifugation step and the pellet will be reconstituted in HBSS (Sigma Aldrich) containing 20% sucrose (Sigma Aldrich). 50 μl will be snapfrozen and used for quantification by a conventional plaque assay. Semi-continuous sucrose gradients will be made in ultracentrifugation grade tubes fitting the SW 41 rotor (Beckman-coulter) by adding a layer of HBSS containing higher percentages of sucrose underneath every layer. Sucrose gradients will start at 30% and will be underlayered with a maximal difference of 5% sucrose, until a percentage of 60% sucrose is reached. Gradients will be incubated at 4° C. for 24 h to allow for mixing and the formation of the semi-continuous sucrose gradient. The reconstituted pellet will be added carefully to the top of the gradients and will be centrifuged for 4 h at 35 000 rpm in the SW41 rotor (Beckman-coulter). Afterwards, tubes will be punctured, and the gradient will be aliquoted per 0.5 ml. Aliquots containing gradient sections between 55% and 35% will be used for quantification by a conventional plaque assay to determine the sucrose percentage containing the highest amount of virus.

### REFERENCES

1. Hall C B, Weinberg G A, Iwane M K, Blumkin A K, Edwards K M, Staat M A, et al. The Burden of Respiratory Syncytial Virus Infection in Young Children. N. Engl. J. Med. [Internet]. 2009;360:588-98. Available from: www.nejm.org/doi/abs/10.1056/NEJMoa0804877.

2. Shi T, Denouel A, Tietjen A K, Campbell I, Moran E, Li X, et al. Global Disease Burden Estimates of Respiratory Syncytial Virus—Associated Acute Respiratory Infection in Older Adults in 2015: A Systematic Review and Meta-Analysis. J. Infect. Dis. [Internet]. 2019;1-8. Available from: academic.oup.com/jid/advance-article/doi/10.1093/infdis/jiz059/5382266.

3. Meng J, Stobart C C, Hotard A L, Moore M L. An Overview of Respiratory Syncytial Virus. PLoS Pathog. 2014.

4. DeFord D M, Nosek J M, Castiglia K R, Hasik E F, Franke M E, Nick B C, et al. Evaluation of the role of respiratory syncytial virus surface glycoproteins F and G on viral stability and replication: implications for future vaccine design. J. Gen. Virol. 2019.

5. Huong T N, Iyer Ravi L, Tan B H, Sugrue R J. Evidence for a biphasic mode of respiratory syncytial virus transmission in permissive HEp2 cell monolayers. Virol. J. [Internet]. Virology Journal; 2016;13:12. Available from: www.virologyj.com/content/13/1/12.

6. Ke Z, Dillard R, Chirkova T, Leon F, Stobart C, Hampton C, et al. The Morphology and Assembly of Respiratory Syncytial Virus Revealed by Cryo-Electron Tomography. Viruses [Internet]. 2018;10:446. Available from: www.mdpi.com/1999-4915/10/8/446.

7. Rostad C A, Stobart C C, Todd S O, Molina S A, Lee S, Blanco J C G, et al. Enhancing the Thermostability and Immunogenicity of a Respiratory Syncytial Virus (RSV) Live-Attenuated Vaccine by Incorporating Unique RSV Line19F Protein Residues. J. Virol. 2017.

8. Van der Gucht W, Leemans A, De Schryver M, Heykers A, Caljon G, Maes L, et al. Respiratory syncytial virus (RSV) entry is inhibited by serine protease inhibitor AEBSF when present during an early stage of infection. Virol. J. [Internet]. Virology Journal; 2017;14:157. Available from: www.ncbi.nlm.nih.gov/pubmed/28818113.

9. Schepens B, Sedeyn K, Vande Ginste L, De Baets S, Schotsaert M, Roose K, et al. Protection and mechanism of action of a novel human respiratory syncytial virus vaccine candidate based on the extracellular domain of small hydrophobic protein. EMBO Mol. Med. [Internet]. 2014;6:1436-54. Available from: embomolmed.embopress.org/cgi/doi/10.15252/emmm.201404005.

10. Houspie L, Lemey P, Keyaerts E, Reijmen E, Vergote V, Vankeerberghen A, et al. Circulation of HRSV in Belgium: From Multiple Genotype Circulation to Prolonged Circulation of Predominant Genotypes. Leung FCC, editor. PLoS One [Internet]. 2013;8:e60416. Available from: www.ncbi.nlm.nih.gov/pubmed/23577109.

11. Leemans A, Boeren M, Van der Gucht W, Pintelon I, Roose K, Schepens B, et al. Removal of the N-Glycosylation Sequon at Position N116 Located in p27 of the Respiratory Syncytial Virus Fusion Protein Elicits Enhanced Antibody Responses after DNA Immunization. Viruses [Internet]. 2018;10:426. Available from: www.mdpi.com/1999-4915/10/8/426.

12. Tapia L I, Shaw C A, Aideyan L O, Jewell A M, Dawson B C, Haq T R, et al. Gene Sequence Variability of the Three Surface Proteins of Human Respiratory Syncytial Virus (HRSV) in Texas. Varga S M, editor. PLoS One [Internet]. 2014;9:e90786. Available from: dx.plos.org/10.1371/j ournal.pone.0090786.

13. Stobart C C, Rostad C A, Ke Z, Dillard R S, Hampton C M, Strauss J D, et al. A live RSV vaccine with engineered thermostability is immunogenic in cotton rats despite high attenuation. Nat. Commun. [Internet]. 2016;7:13916. Available from: www.nature.com/articles/ncomms13916.

14. Gagliardi T B, Criado M F, Proenca-Módena J L, Saranzo A M, Iwamoto M A, de Paula F E, et al. Syncytia Induction by Clinical Isolates of Human Respiratory Syncytial Virus A. Intervirology [Internet]. 2017;60:56-60. Available from: www.karger.com/Article/FullText/480014.

15. Ridley C, Thornton D J. Mucins: the frontline defence of the lung. Biochem. Soc. Trans. [Internet]. 2018;46:1099-106. Available from: www.biochemsoctrans.org/cgi/doi/10.1042/BST20170402.

16. Baños-Lara M D R, Piao B, Guerrero-Plata A. Differential Mucin Expression by Respiratory Syncytial Virus and Human Metapneumovirus Infection in Human Epithelial Cells. Mediators Inflamm. [Internet]. 2015;2015:1-7. Available from: www.hindawi.com/journals/mi/2015/347292/.

17. Justicia-Grande A J, Pardo-Seco J, Cebey-López M, Vilanova-Trillo L, Gómez-Carballa A, Rivero-Calle I, et al. Development and validation of a new clinical scale for infants with acute respiratory infection: The resvinet scale. PLoS One. 2016;11:1-15.

18. Hotard A L, Shaikh F Y, Lee S, Yan D, Teng M N, Plemper R K, et al. A stabilized respiratory syncytial virus reverse genetics system amenable to recombination-mediated mutagenesis. Virology [Internet]. 2012;434:129-36. Available from: linkinghub.elsevier.com/retrieve/pii/50042682212004631.

1-16. (canceled)

17. A method of diagnosing and treating a patient with a BE/ANT-A11/17 strain of respiratory syncytial virus (RSV), the method comprising:
isolating a virus from a biological sample obtained from the patient;
exposing a cell line to the virus, wherein the cell-line is selected from the group consisting of HEp-2, A549, Vero, and BEAS-2B;
culturing the cell line with the virus, thereby replicating the virus;
isolating the amplified virus from the cell line by collecting a volume of supernatant;
characterizing the isolated virus to generate a viral profile, wherein the viral profile comprises data collected from one or more of a G-protein sequence analysis, a viral replication kinetic analysis, a thermal stability assay, a MUC4 expression assay, a MUC5B expression assay, an F gene sequence analysis, or a whole genome sequencing analysis;
diagnosing the patient as being infected with the BE/ANT-A11/17 strain of RSV when the viral profile matches a known profile of the BE/ANT-A11/17 strain and is compared to a control profile; and
treating the diagnosed patient with a monoclonal antibody that binds BE/ANT-A 11/17.

18. The method of claim 17, wherein the monoclonal antibody is palivizumab.

19. The method of claim 17, wherein the monoclonal antibody is a human anti-BE/ANT-A11/17 antibody.

20. The method of claim 17, wherein the control profile is a profile of an RSV A2 strain of RSV.

21. The method of claim 17, wherein the G-protein sequence analysis comprises:
reverse transcribing viral RNA corresponding to a G-protein in to cDNA;
amplifying the cDNA using a polymerase chain reaction with a forward primer as set forth in SEQ ID NO:7 or SEQ ID NO:9 and a reverse primer as set forth in SEQ ID NO:8 or SEQ ID NO:10; and
sequencing the cDNA using a forward primer as set forth in SEQ ID NO:11 or SEQ ID NO:13 and a reverse primer as set forth in SEQ ID NO:12 or SEQ ID NO:14.

22. The method of claim 17, wherein viral replication kinetic analysis comprises:
culturing a second cell line with the virus for 2 hours, wherein the second cell line is selected from the group consisting of HEp-2, A549, and BEAS-2B;
fixing the second cell line with paraformaldehyde after a given time period after culturing the second cell line;
staining and permeabilizing the fixed cell line with palivizumab;
staining the fixed cell line with goat anti-human secondary antibody conjugated with Alexa Fluor 488; and
analyzing the stained cell line using fluoresce microscopy,
wherein an increased rate of viral replication compared to a control is associated with the BE/ANT-A11/17 strain of RSV.

23. The method of claim 17, wherein the thermal stability assay comprises:
aliquoting the volume of supernatant into a plurality of samples;
snap freezing a first sample in liquid nitrogen;
storing a second sample at 4 degrees Celsius for a time period selected from 24 hours, 48 hours, or 72 hours;
snap freezing the second sample in liquid nitrogen after the time period has passed;
storing the remaining samples at temperatures selected from 4 degrees Celsius, 32 degrees Celsius, or 37 degrees Celsius for time periods selected from 24 hours, 48 hours, or 72 hours;
snap freezing the remaining sample in liquid nitrogen after the time periods have passed; and
performing a plaque assay on the snap frozen samples to quantify a conservation of PFUs in each sample.

24. The method of claim 23, wherein an increased PFU conservation at 4 degrees Celsius compared to the control profile is associated with the BE/ANT-A11/17 strain of RSV.

25. The method of claim 17, wherein the MUC4 expression assay comprises:
lysing the cell line remaining after supernatant collection;
isolating RNA from the lysed cells;
reverse transcribing the isolated RNA into cDNA; and
quantifying MUC4 expression levels using a polymerase chain reaction,
wherein an increased MUC4 expression level compared to a control profile is associated with the BE/ANT-A11/17 strain of RSV.

26. The method of claim 17, wherein the MUCSB expression assay comprises:
lysing the cell line remaining after supernatant collection;
isolating RNA from the lysed cells;
reverse transcribing the isolated RNA into cDNA; and
quantifying MUCSB expression levels using a polymerase chain reaction,
wherein a decreased MUC5B expression level compared to a control profile is associated with the BE/ANT-A11/17 strain of RSV.

27. The method of claim 17, wherein the F-protein sequence analysis comprises:
reverse transcribing viral RNA corresponding to an F-protein in to cDNA;
amplifying the cDNA using a polymerase chain reaction with a forward primer as set forth in SEQ ID NO:15 and SEQ ID NO:17 and a reverse primer as set forth in SEQ ID NO:16 and SEQ ID NO:18; and
verifying the length of the amplified cDNA using gel electrophoresis; and
sequencing the cDNA using the forward primer as set forth in SEQ ID NO:15 and SEQ ID NO:17 and the reverse primer as set forth in SEQ ID NO:16 and SEQ ID NO:18.

28. The method of claim 27, wherein the F-protein sequence comprises mutations resulting in changes in at least 12 amino acids compared to the control profile, the changes occurring in a signal peptide, a fusion peptide, and F1 subunit, and F2 subunit, an HRB region, and a transmembrane domain.

29. The method of claim 28, wherein the F-protein sequence further comprises a substitution of A to T in the p27 at residue 122 compared to the control profile, resulting in an N-glycosylation site, wherein the substitution is associated with the BE/ANT-A11/17 strain of RSV.

30. The method of claim 17, wherein the whole genome sequencing analysis comprises:
extracting the viral RNA;
reverse transcribing the viral RNA into cDNA;
synthesizing a complementary strand of DNA;
amplifying the double stranded DNA using a polymerase chain reaction; and
sequencing the DNA using pair end sequencing.

31. A method of producing cell-free RSV for use in formulating a vaccine, the method comprising:
providing a cell line selected from the group consisting of HEp-2, A549, Vero, and BEAS-2B;
infecting the cell line with the virus or transfecting the cell line with a plasmid carrying a nucleic acid sequence encoding the virus, wherein the virus is deposited at Belgian Coordinated Collection of Micro-Organisms under deposit number LMBP 11505;
culturing the cell line infected or transfected, thereby producing the virus; and
isolating the cell-free RSV from the cell culture.


SEQUENCE LISTING (18 sequences)

SEQ ID NO: 1 - DNA, 26 bp, Artificial Sequence
  RSVQA1 primer
gctcttagcaaagtcaagttgaatga

SEQ ID NO: 2 - DNA, 21 bp, Artificial Sequence
  RSVQA2 primer
tgctccgttggatggtgtaat

SEQ ID NO: 3 - DNA, 33 bp, Artificial Sequence
  RSVQA probe
acactcaacaaagatcaacttctgtcatccagc

SEQ ID NO: 4 - DNA, 26 bp, Artificial Sequence
  RSVQB1 primer
gatggctcttagcaaagtcaagttaa

SEQ ID NO: 5 - DNA, 30 bp, Artificial Sequence
  RSVQB2 primer
tgtcaatattatctcctgtactacgttgaa

SEQ ID NO: 6 - DNA, 35 bp, Artificial Sequence
  RSVQB probe
tgatacattaaataaggatcagctgctgtcatcca

SEQ ID NO: 7 - DNA, 20 bp, Artificial Sequence
  G267FW fw primer
atgcaacaagccagatcaag

SEQ ID NO: 8 - DNA, 23 bp, Artificial Sequence
  F164RV rv primer
gttatcacactggtataccaacc

SEQ ID NO: 9 - DNA, 24 bp, Artificial Sequence
  BGF Fw primer
gcagccataatattcatcatctct

SEQ ID NO: 10 - DNA, 22 bp, Artificial Sequence
  BGR rv primer
tgccccagrtttaatttcgttc

SEQ ID NO: 11 - DNA, 22 bp, Artificial Sequence
  G516R primer
gctgcagggtacaaagttgaac

SEQ ID NO: 12 - DNA, 20 bp, Artificial Sequence
  G284F primer
acctgacccagaatccccag

SEQ ID NO: 13 - DNA, 23 bp, Artificial Sequence
  BGF3 primer
agagacccaaaaacacyagccaa

SEQ ID NO: 14 - DNA, 25 bp, Artificial Sequence
  BGR3 primer
acagggaacgaagttgaacacttca

SEQ ID NO: 15 - DNA, 20 bp, Artificial Sequence
  RSVAB_F1FW primer
ggcaaataacaatggagttg

SEQ ID NO: 16 - DNA, 19 bp, Artificial Sequence
  RSVAB_F1RV
aagaaagatactgatcctg

SEQ ID NO: 17 - DNA, 20 bp, Artificial Sequence
  RSVAB_F2FW
tcaatgatatgcctataaca

SEQ ID NO: 18 - DNA, 22 bp, Artificial Sequence
  RSVAB_F2RV
ggacattacaaataattatgac